(19) United States
(12) Patent Application Publication
(10) Pub. No.: US 2023/0151345 A1
(43) Pub. Date: May 18, 2023

(54) COMPOSITIONS AND METHODS FOR ENHANCING HOMOLOGOUS RECOMBINATION

(71) Applicant: LIFE TECHNOLOGIES CORPORATION, Carlsbad, CA (US)

(72) Inventors: Robert POTTER, San Marcos, CA (US); Jonathan CHESNUT, Carlsbad, CA (US); Xiquan LIANG, Escondido, CA (US)

(21) Appl. No.: 18/071,206

(22) Filed: Nov. 29, 2022

Related U.S. Application Data
(62) Division of application No. 16/534,636, filed on Aug. 7, 2019.
Application No. 16/534,636 is a division of application No. 15/520,533, filed on Apr. 20, 2017, now abandoned.
(60) Provisional application No. 62/068,451, filed on Oct. 24, 2014.

(51) Int. Cl. C12N 9/22; C12N 15/11; C12N 15/90

(52) U.S. Cl. CPC C12N 9/22; C12N 15/11; C12N 15/907; C12N 2310/20; C12N 2800/80

(57) ABSTRACT

The present disclosure generally relates to compositions and methods for improving the efficiency of homologous recombination. In particular, the disclosure relates to reagents and the use of such reagents.

### CROSS-REFERENCE TO RELATED APPLICATIONS

This application is a divisional of U.S. patent application Ser. No. 16/534,636, filed on Aug. 7, 2019, which is a divisional of U.S. patent application Ser. No. 15/520,533 filed on Apr. 20, 2017, now abandoned, which is a 371 National Phase Application of International Application No. PCT/US2015/057401 filed on Oct. 26, 2015, now expired, which claims the benefit of prior to U.S. Provisional Patent Application No. 62/068,451 filed on Oct. 24, 2014, now expired, which disclosures are herein incorporated by reference in their entirety.

### SEQUENCE LISTING

The instant application contains a Sequence Listing which has been submitted electronically in ASCII format and is hereby incorporated by reference in its entirety. Said ASCII copy, created on Dec. 20, 2022, is named TP103220USDIV2_SL.xml and is 10,570 bytes in size.

### FIELD

The present disclosure generally relates to compositions and methods for improving the efficiency of homologous recombination. In particular, the disclosure relates to reagents and the use of such reagents.

### BACKGROUND

A number of genome-editing systems, such as designer zinc fingers, transcription activator-like effectors (TALEs), CRISPRs, and homing meganucleases, have been developed. One issue with these systems is low levels of homologous recombination often requires that numerous cells of clonal origin be screened to identify cells that have undergone homologous recombination and have the desired genotype. The generation and identification of cells with the correct genotype is often laborious and time consuming. In one aspect, the invention allows for the efficient design, preparation, and use of genome editing reagents and generation and identification of cells that have been “correctly” edited.

### SUMMARY

The present disclosure relates, in part, to compositions and methods for editing of nucleic acid molecules. There exists a substantial need for efficient systems and techniques for modifying genomes. This invention addresses this need and provides related advantages.

One aspect of the invention involves enhancing homologous recombination by increasing the concentration of donor nucleic acid at or in close proximity to the junction of a break in a nucleic acid molecule resident in a cell (e.g., a chromosome).

In specific instances, the disclosure relates to the following clauses:

Clause 1: A method for the introduction of a donor nucleic acid molecule into a target locus present in a cell, the method comprising introducing into the cell a nucleic acid cutting entity associated with the donor nucleic acid molecule, wherein the nucleic acid cutting entity generates a double stranded break in nucleic acid present in the cell, and wherein the donor nucleic acid molecule is brought into close proximity to the double stranded break by association with the nucleic acid cutting entity.

Clause 2: The method of clause 1, wherein the nucleic acid cutting entity is selected from the group consisting of: (a) a zinc finger nuclease fusion, (b) a TAL effector nuclease fusion, and (c) a CRISPR complex.

Clause 3: The method of clause 1, wherein the donor nucleic acid molecule is covalently bound to at least one component of the nucleic acid cutting entity.

Clause 4: The method of clause 1, wherein the double stranded break in nucleic acid present in the cell generated by the nucleic acid cutting entity is produced by the homodimerization of two FokI nuclease domains, where each FokI nuclease domain is covalently bound to different protein molecules.

Clause 5: The method of clause 2, wherein the nucleic acid cutting entity is a TAL effector.

Clause 6: The method of clause 1, wherein greater than 25% of target loci that have undergone double stranded breaks incorporate the donor nucleic acid.

Clause 7: A method for enhancing homologous recombination at a target locus of a nucleic acid molecule in cells, the method comprising: (a) introducing into the cells a nucleic acid cutting entity associated with a donor nucleic acid molecule, and (b) obtaining cells that have undergone homologous recombination and non-homologous end joining, wherein the number of cells that have undergone homologous recombination is at least 5 fold higher than the number of cells that have undergone non-homologous end joining.

Clause 8: The method of clause 7, wherein the donor nucleic acid molecule is from about 50 nucleotides to about 10,000 nucleotides in length.

Clause 9: The method of clause 7, wherein the donor nucleic acid molecule contains two region of sequence homology to nucleic acid at the target locus, wherein each region of sequence homology is from about 25 nucleotides to about 400 nucleotides in length.

Clause 10: The method of clause 7, wherein the donor nucleic acid molecule contains a selectable marker.

Clause 11: A composition comprising a component of a nucleic acid cutting entity, wherein a donor nucleic acid molecules is associated with the nucleic acid cutting entity.

Clause 12: The composition of clause 11, wherein the donor nucleic acid molecules is covalently bound to at least one component of the nucleic acid cutting entity.

Clause 13: A composition comprising a CRISPR RNA molecule and a donor nucleic acid molecule, wherein the donor nucleic acid molecule is covalently bound to the CRISPR RNA molecule.

Clause 14: The composition of clause 13, wherein the donor nucleic acid molecule is covalently bound to a guide RNA molecule.

Clause 15: The composition of clause 14, wherein the donor nucleic acid molecule is covalently bound to the 3′ terminus of the guide RNA molecule.

Clause 16: The composition of clause 13, wherein the donor nucleic acid molecule is covalently bound to a tracer RNA molecule.

Clause 17: The composition of clause 13, further comprising a transfection reagent.

Clause 18: A composition comprising a Cas9 protein and a donor nucleic acid molecule, wherein the donor nucleic acid molecule is bound to the Cas9 protein.

Clause 19: The composition of clause 17, wherein the donor nucleic acid molecule is non covalently bound to the Cas9 protein.

Clause 20: The composition of clause 19, wherein the donor nucleic acid molecule contains a biotin moiety, the Cas9 protein contains and avidin group, and the donor nucleic acid molecule and Cas9 protein are associated with each other through an interaction between biotin and avidin.

### BRIEF DESCRIPTION OF THE DRAWINGS

For a more complete understanding of the principles disclosed herein, and the advantages thereof, reference is made to the following descriptions taken in conjunction with the accompanying drawings, in which:

FIG. 1 is a representative diagram showing some variations of the invention. A: The black boxes represent two zinc finger nucleases with cleavage specificity for the same locus of a host cell chromosome. The open circle indicates a linkage point, and the wiggly line to the right of the connection point represents donor DNA. B: The shaded boxes represent two TAL effector nucleases with cleavage specificity for the same locus of a host cell chromosome. Other representations in this Panel and in Panels C, D, E, and F are the same as in Panel A. C, D, E, and F: The shaded circles represent Cas9 protein. The hairpin nucleic acid molecule is guide RNA. In C, donor DNA is linked only to guide RNA. In D, donor DNA is linked to Cas9 protein and guide RNA. Also, the represented Cas9 protein has two donor nucleic acid molecules linked to it. In E, donor DNA is linked only to Cas9 protein. In F, donor DNA is linked to two Cas9 proteins. These Cas9 proteins have mutations (e.g., in the HNH and RuvC domain) that result in each protein having nickase activity instead of double-stranded cleavage activity.

FIG. 2 shows two exemplary donor nucleic acid molecules (i.e., “Construct 1” and “Construct 2”) designed to introduce an insert (in white) into a nucleic acid molecules resident in a cell by homologous recombination. Both constructs have donor homology regions on each side of an insert region (in black). Construct 1 shows (in grey) a flanking region located on the left side of the construct. The lower portion of this figure shows a chromosomal locus containing a double-stranded break. The donor homology regions of Construct 2 are indicated as undergoing homologous recombination with their corresponding regions in at the chromosomal locus (e.g., chromosomal nucleic acid on each side of the target locus, labeled “Chromosomal Locus 1” and “Chromosomal Locus 2”).

FIG. 3 shows an overview of one possible mechanism by which nucleic acid cutting entity nucleic acid is brought into close proximity with nucleic acid at a target locus. Labels are as in FIG. 2. In this instance, donor nucleic acid is linked to a TAL effector protein through a linking group.

FIG. 4 shows an exemplary method for linking an RNA segment to a DNA segment. The linking reaction shown in this figure using propargyl on one terminus and azide on the other terminus is unidirectional in that the termini with the chemical modifications are the only one that can link with each other.

FIG. 5 shows an exemplary method for linking a protein molecule to a DNA segment.

FIG. 6 shows a method for quantitation of homologous recombination. The Donor DNA contains EcoRI restriction sites as indicated. Fo and Ro indicate the forward and reverse primers, located outside of the donor fragment. Rr and Rt primers are designed to give PCR fragments derived from a successfully integrated donor DNA. PCR fragments amplified with Fo/Ro are digested with EcoRI, followed by agar gel separation. The percentages of digested bands, quantified with ALPHAIMAGER®, represent the homologous recombination efficiency.

FIG. 7 Shows step 1 of the synthesis of gRNA-azido-dATP. gRNA is incubated with azido-dATP in the presence of Poly(A) Polymerase (SEQ ID NOS 7 and 7, respectively, in order of appearance).

FIG. 8 Shows step 2 of the synthesis of alkyne-ssDNA or alkyne-dsDNA. 5′ or 3′-amine modified single strand or double strand DNA molecules are coupled to amine-reactive alkyne, succinimidyl ester.

FIG. 9 Shows coupling of gRNA to ss or ds DNA using Click chemistry (SEQ ID NOS 7 and 7, respectively, in order of appearance).

FIG. 10(A) Shows the gel analysis of the PCR product obtained from the Jurkat T cells transfected with Cas9 protein and 250 or 500 ng of gRNA/dsDonor conjugate, dsDonor or gRNA, respectively. The PCR products are subjected to EcoRI digestion. +/− indicates the presence or absence of the corresponding component in the reaction. FIG. 10(B) Shows the sequencing analysis of the PCR product and the relative distribution of the various products based on the sequence analysis.

FIG. 11 Shows the gel analysis of the PCR product obtained from the Jurkat T cells transfected with Cas9 protein and 200 or 500 ng of gRNA/dsDonor conjugate, gRNA/ssDonor conjugate, dsDonor, ssDonor or gRNA, respectively. The products are subjected to EcoRI disgestion. +/ indicates the presence or absence of the corresponding component in the reaction.

### DETAILED DESCRIPTION

### Definitions

As used herein the term “homologous recombination” refers to a mechanism of genetic recombination in which two DNA strands comprising similar nucleotide sequences exchange genetic material. Cells use homologous recombination during meiosis, where it serves to rearrange DNA to create an entirely unique set of haploid chromosomes, but also for the repair of damaged DNA, in particular for the repair of double strand breaks. The mechanism of homologous recombination is well known to the skilled person and has been described, for example by Paques and Haber (Paques F, Haber J E.; Microbiol. Mol. Biol. Rev. 63:349-404 (1999)). In the method of the present invention, homologous recombination is enabled by the presence of said first and said second flanking element being placed upstream (5′) and downstream (3′), respectively, of said donor DNA sequence each of which being homologous to a continuous DNA sequence within said target sequence.

As used herein the term “non-homologous end joining” (NEHJ) refers to cellular processes that join the two ends of double-strand breaks (DSBs) through a process largely independent of homology. Naturally occurring DSBs are generated spontaneously during DNA synthesis when the replication fork encounters a damaged template and during certain specialized cellular processes, including V(D)J recombination, class-switch recombination at the immunoglobulin heavy chain (IgH) locus and meiosis. In addition, exposure of cells to ionizing radiation (X-rays and gamma rays), UV light, topoisomerase poisons or radiomimetic drugs can produce DSBs. NHEJ (non-homologous end-joining) pathways join the two ends of a DSB through a process largely independent of homology. Depending on the specific sequences and chemical modifications generated at the DSB, NHEJ may be precise or mutagenic (Lieber M R., The mechanism of double-strand DNA break repair by the nonhomologous DNA end-joining pathway. Annu Rev Biochem 79:181-211).

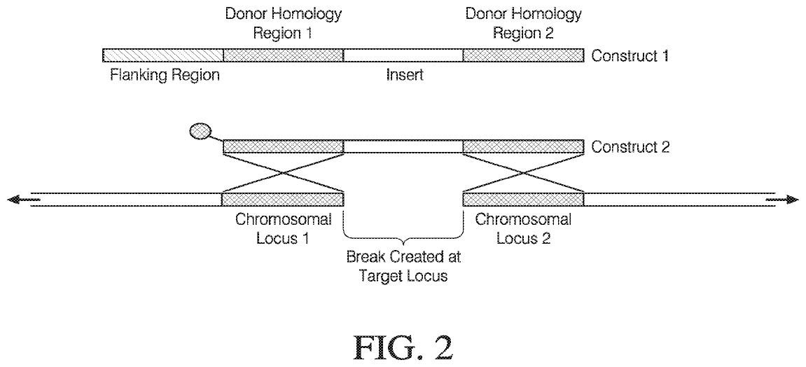

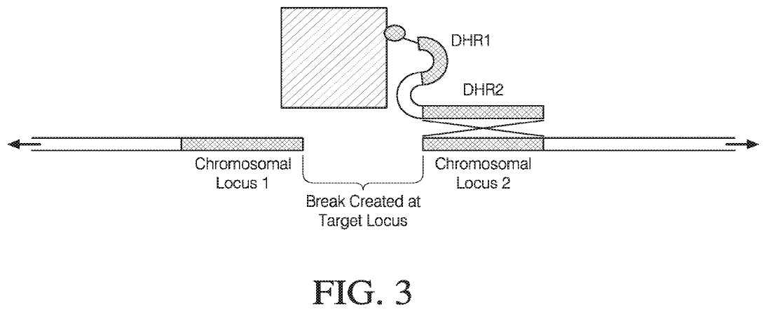

As used herein the term “donor DNA” or “donor nucleic acid” refers to nucleic acid that is designed to be introduced into a locus by homologous recombination. Donor nucleic acid will have at least one region of sequence homology to the locus. In many instances, donor nucleic acid will have two regions of sequence homology to the locus. These regions of homology may be at one of both termini or may be internal to the donor nucleic acid. In many instances, and “insert” region with nucleic acid that one desires to be introduced into a nucleic acid molecules present in a cell will be located between two regions of homology (see FIG. 2).

As used herein the term “homologous recombination system or “HR system” refers components of systems set out herein that maybe used to alter cells by homologous recombination. In particular, zinc fingers, TAL effectors, and CRISPR systems.

As used herein the term “nucleic acid cutting entity” refers to a single molecule or a complex of molecules that has nucleic acid cutting activity (e.g., double-stranded nucleic acid cutting activity). Exemplary nucleic acid cutting entities include zinc fingers, transcription activator-like effectors (TALEs), CRISPRs, and homing meganucleases.

As used herein the term “zinc finger protein (ZFP)” refers to a protein comprising refers to a polypeptide having nucleic acid (e.g., DNA) binding domains that are stabilized by zinc. The individual DNA binding domains are typically referred to as “fingers,” such that a zinc finger protein or polypeptide has at least one finger, more typically two fingers, or three fingers, or even four or five fingers, to at least six or more fingers. In some aspect, ZFPs will contain three or four zinc fingers. Each finger typically binds from two to four base pairs of DNA. Each finger usually comprises an about 30 amino acids zinc-chelating, DNA-binding region (see, e.g., U.S. Pat. Publ. No. 2012/0329067 A1, the disclosure of which is incorporated herein by reference).

In many instances, zinc finger proteins will contain nuclear localization signals (NLS) that allow them to be transported to the nucleus.

As used herein the term “transcription activator-like effectors (TAL)” refers to proteins composed of more than one TAL repeat and is capable of binding to nucleic acid in a sequence specific manner. In many instances, TAL effectors will contain at least six (e.g., at least 8, at least 10, at least 12, at least 15, at least 17, from about 6 to about 25, from about 6 to about 35, from about 8 to about 25, from about 10 to about 25, from about 12 to about 25, from about 8 to about 22, from about 10 to about 22, from about 12 to about 22, from about 6 to about 20, from about 8 to about 20, from about 10 to about 22, from about 12 to about 20, from about 6 to about 18, from about 10 to about 18, from about 12 to about 18, etc.) TAL repeats. In some instances, a TAL effector may contain 18 or 24 or 17.5 or 23.5 TAL nucleic acid binding cassettes. In additional instances, a TAL effector may contain 15.5, 16.5, 18.5, 19.5, 20.5, 21.5, 22.5 or 24.5 TAL nucleic acid binding cassettes. TAL effectors will generally have at least one polypeptide region which flanks the region containing the TAL repeats. In many instances, flanking regions will be present at both the amino and carboxyl termini of the TAL repeats. Exemplary TALs are set out in U.S. Pat. Publ. No. 2013/0274129 A1 and may be modified forms on naturally occurring proteins found in bacteria of the genera Burkholderia, Xanthamonas and Ralstonia.

In many instances, TAL proteins will contain nuclear localization signals (NLS) that allow them to be transported to the nucleus.

As used herein the term “CRISPR complex” refers to the CRISPR proteins and nucleic acid (e.g., RNA) that associate with each other to form an aggregate that has functional activity. An example of a CRISPR complex is a wild-type Cas9 (sometimes referred to as Csn1) protein that is bound to a guide RNA specific for a target locus.

As used herein the term “CRISPR protein” refers to a protein comprising a nucleic acid (e.g., RNA) binding domain nucleic acid and an effector domain (e.g., Cas9, such as Streptococcus pyogenes Cas9). The nucleic acid binding domains interact with a first nucleic acid molecules either having a region capable of hybridizing to a desired target nucleic acid (e.g., a guide RNA) or allows for the association with a second nucleic acid having a region capable of hybridizing to the desired target nucleic acid (e.g., a crRNA). CRISPR proteins can also comprise nuclease domains (i.e., DNase or RNase domains), additional DNA binding domains, helicase domains, protein-protein interaction domains, dimerization domains, as well as other domains.

CRISPR protein also refers to proteins that form a complex that binds the first nucleic acid molecule referred to above. Thus, one CRISPR protein may bind to, for example, a guide RNA and another protein may have endonuclease activity. These are all considered to be CRISPR proteins because they function as part of a complex that performs the same functions as a single protein such as Cas9.

In many instances, CRISPR proteins will contain nuclear localization signals (NLS) that allow them to be transported to the nucleus.

As used herein the term “target locus” refers to a site within a nucleic acid molecule that is recognized and cleavage by a nucleic acid cutting entity. When, for example, a single CRISPR complex is designed to cleave double-stranded nucleic acid, then the target locus is the cut site and the surrounding region recognized by the CRISPR complex. When, for example, two CRISPR complexes are designed to nick double-stranded nucleic acid in close proximity to create a double-stranded break, then the region surrounding recognized by both CRISPR complexes and including the break point is referred to as the target locus.

### Overview

The invention relates, in part, to (1) components of nucleic acid cutting entities that contain one or more exogenous linking group, (2) donor nucleic acid molecules that contain one or more exogenous linking group (e.g., a linking group that is not a group normally found in DNA and RNA), (3) compositions comprising nucleic acid cutting entity associated with (e.g., covalently bound, non-covalently bound, etc.) one or more donor nucleic acid molecules, and (4) methods for using components and methods set out herein for performing homologous recombination.

The invention relates, in part, to compositions and methods for enhancing homologous recombination reactions. The invention also related, in part, to increasing the homologous recombination (HR) to non-homologous end-joining (NHEJ) ratio. Both of these aspects of the invention may be achieved by the delivery of donor nucleic acid to a target locus by associating it with one or more nucleic acid cutting entities. While not wishing to be bound to theory, it is believed that both increased HR efficiency and increased HR as compared to NHEJ are the result of a high local concentration of donor nucleic acid at target loci that have a double-stranded break.

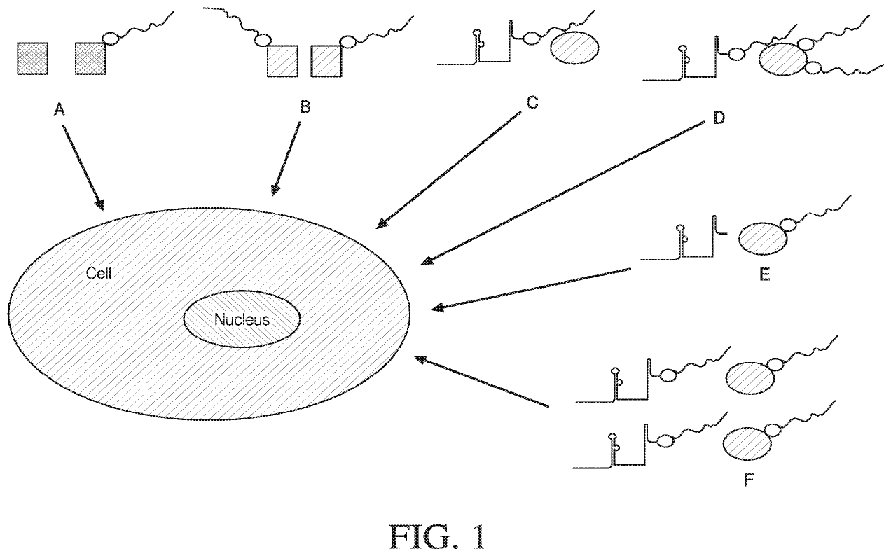

In most instances, methods of the invention employ at least one donor nucleic acid that is associated with at least one component of a nucleic acid cutting entity. Examples of some embodiments of compositions and methods of the invention are set out in FIG. 1. Panel A of FIG. 1 shows two zinc finger nucleases (e.g., zinc finger-FokI fusions) designed to cut the same target locus. A donor nucleic acid molecule is covalently bound to one of the two zinc finger nucleases via a linkage site. Panel B of FIG. 1 shows two TALs (e.g., TAL-FokI fusions) designed to cut the same target locus but, in this instance, each of the TALs has a covalently bound donor nucleic acid molecule.

Panels C, D, E, and F show four different variations of CRISPR systems. In each instance, donor nucleic acid is covalently linked to guide RNA (C), a CRISPR protein (e.g., Cas9) (E), or both (D). crRNA and tracrRNA may be employed instead of guide RNA, with donor nucleic acid being associated with one or both of thee RNA molecules.

In some instances, two CRISPR complexes targeting the same target locus may each contain two donor nucleic acids (e.g., Panel F of FIG. 1). This would result in four donor nucleic acid molecules being brought into close proximity to a single target locus.

### Donor Nucleic Acid

Donor nucleic acids will typically contain regions of homology corresponding to nucleic acid surrounding a target locus. Two exemplary donor nucleic acids are set out in FIG. 2 as Construct 1 and Construct 2.

Construct 1 and Construct 2 have three regions in common. The two donor homology regions (black) flank an insert (white) and are designed to undergo homologous recombination with nucleic acid on each side of a target locus that has undergone a double-stranded break.

Construct 1 also has a flanking region that is not located between the two donor homology regions (grey). In many instances, the flanking region will encode a negative selection marker (e.g., Herpes simplex thymidine kinase, HPRT, GPT, Diphtheria toxin, etc.). The purpose of this marker is select against cells in which Construct 1 has randomly integrated into a cells genome. In most instances, when Construct 1 is introduced into a cellular genome by HR, any nucleic acid outside of the donor homology regions will not be introduced into the genome. Nucleic acid constructs such as Construct 1, and methods for using such constructs are set out in Capecchi et al., U.S. Pat. No. 5,464,764, the disclosure of which is incorporated herein by reference. Thus, the invention includes compositions and methods for the introduction of donor nucleic acids into cell that have a negative selection marker. The invention further includes compositions and methods for the selection of cells, using such markers, to obtain a population of cells that have introduced donor nucleic acid via homologous recombination.

The homology regions may be of varying lengths and may have varying amounts of sequence identity with nucleic acid at the target locus. Typically, homologous recombination efficiency increases with increased lengths and sequence identity of homology regions. The length of homology regions employed is often determined by factors such as fragility of large nucleic acid molecules, transfection efficiency, and ease of generation of nucleic acid molecules containing homology regions.

While the length of two homology regions within the same donor nucleic acid may be the same or different, homology regions may be from about 40 bases to about 10,000 bases in total length (e.g., from about 50 bases to about 8,000 bases, from about 50 bases to about 7,000 bases, from about 50 bases to about 6,000 bases, from about 50 bases to about 5,000 bases, from about 50 bases to about 3,000 bases, from about 50 bases to about 2,000 bases, from about 50 bases to about 1,000 bases, from about 50 bases to about 800 bases, from about 50 bases to about 600 bases, from about 50 bases to about 500 bases, from about 50 bases to about 400 bases, from about 50 bases to about 300 bases, from about 50 bases to about 200 bases, from about 100 bases to about 8,000 bases, from about 100 bases to about 2,000 bases, from about 100 bases to about 1,000 bases, from about 100 bases to about 700 bases, from about 100 bases to about 600 bases, from about 100 bases to about 400 bases, from about 100 bases to about 300 bases, from about 150 bases to about 8,000 bases, from about 150 bases to about 1,000 bases, from about 150 bases to about 500 bases, from about 150 bases to about 400 bases, from about 200 bases to about 8,000 bases, from about 200 bases to about 1,000 bases, from about 200 bases to about 600 bases, from about 200 bases to about 400 bases, from about 200 bases to about 300 bases, from about 250 bases to about 8,000 bases, from about 250 bases to about 2,000 bases, from about 250 bases to about 1,000 bases, from about 350 bases to about 8,000 bases, from about 350 bases to about 2,000 bases, from about 350 bases to about 1,000 bases, etc.).

The amount of sequence identity the homologous regions share with the nucleic acid at the target locus, typically the higher the homologous recombination efficiency. High levels of sequence identity are especially desired when the homologous regions are fairly short (e.g., 50 bases). Typically, the amount of sequence identity between the target locus and the homologous regions will be greater than 90% (e.g., from about 90% to about 100%, from about 90% to about 99%, from about 90% to about 98%, from about 95% to about 100%, from about 95% to about 99%, from about 95% to about 98%, from about 97% to about 100%, etc.).

As used herein, “percentage of sequence identity” means the value determined by comparing two optimally aligned nucleotide sequences over a comparison window, wherein the portion of the nucleotide sequence in the comparison window may comprise additions or deletions (i.e., sequence alignment gaps) as compared to the reference sequence (which does not comprise additions or deletions) for optimal alignment of the two sequences. In other words, sequence alignment gaps are removed for quantification purposes. The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The invention also provide compositions and methods for the introduction into intracellular nucleic acid of a small number of bases (e.g., from about 1 to about 10, from about 1 to about 6, from about 1 to about 5, from about 1 to about 2, from about 2 to about 10, from about 2 to about 6, from about 3 to about 8, etc.). For purposes of illustration, a donor nucleic acid molecule may be prepared that is fifty-one bases pairs in length. This donor nucleic acid molecule may have two homology regions that are 25 base pairs in length with the insert region being a single base pair. When nucleic acid surrounding the target locus essentially matches the regions of homology with no intervening base pairs, homologous recombination will result in the introduction of a single base pair at the target locus. Homologous recombination reactions such as this can be employed, for example, to disrupt protein coding reading frames, resulting in the introduction of a frame shift in intracellular nucleic acid. The invention thus provides compositions and methods for the introduction of one or a small number of bases into intracellular nucleic acid molecules.

The invention further provides compositions and methods for the alteration of short nucleotide sequences in intracellular nucleic acid molecules. One example of this would be the change of a single nucleotide position, with one example being the correction or alteration of a single-nucleotide polymorphism (SNP). Using SNP alteration for purposes of illustration, a donor nucleic acid molecule may be designed with two homology regions that are 25 base pairs in length. Located between these regions of homology is a single base pair that is essentially a “mismatch” for the corresponding base pair in the intracellular nucleic acid molecules. Thus, homologous recombination may be employed to alter the SNP by changing the base pair to either one that is considered to be wild-type or to another base (e.g., a different SNP). Cells that have correctly undergone homologous recombination may be identified by later sequencing of the target locus.

One method for determining sequence identity values is through the use of the BLAST 2.0 suite of programs using default parameters (Altschul et al., Nucleic Acids Res. 25:3389-3402 (1997)). Software for performing BLAST analyses is publicly available, e.g., through the National Center for Biotechnology-Information.

Donor nucleic acid may also contain elements desired for insertion (i.e., an insert) into an intracellular nucleic acid molecule (e.g., a chromosome or plasmid) by homologous recombination. Such elements may be selectable markers (e.g., a positive selectable marker such as an antibiotic resistance marker), promoter elements, non-selectable marker protein coding nucleic acid (e.g., nucleic acid encoding cytokines, growth factors, etc.). Inserts may also encode detectable proteins such as luciferase and fluorescent proteins such as green fluorescent protein and yellow fluorescent protein).

Donor nucleic acid will typically be DNA and may be single-stranded or double-stranded. Further, donor nucleic acid may also contain one or more linking group used to connect the donor nucleic acid to either protein or other nucleic acids (e.g., a guide RNA molecule). Linking groups may be located at a 3′ terminus, a 5′ terminus, and/or interior in donor nucleic acids. Thus, the invention includes compositions comprising nucleic acid molecules and proteins that contain one or more linking group. As an example, the invention includes compositions comprising a donor nucleic acid molecule with linking group and one or more of the following: (1) a protein that contains one or more cognate linking group and (2) another nucleic acid molecule that one or more cognate linking group. The invention further includes compositions comprising one or more donor nucleic acid molecule linked to a protein or another nucleic acid molecule. In most instance, the protein and/or the another nucleic acid molecule will be a component of a nucleic acid cutting entity, or associated with a nucleic acid cutting entity.

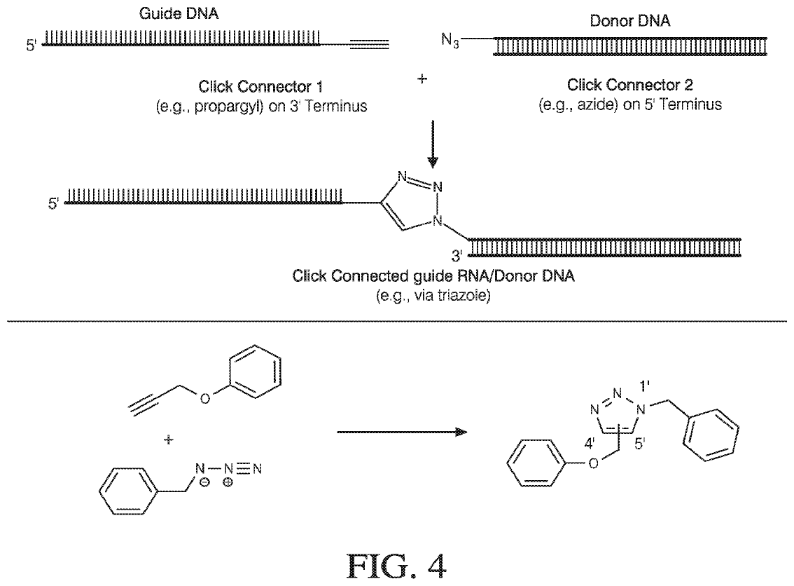

As used herein, the term “cognate linking groups” refers to two linking groups that are capable of binding to each other with sufficient affinity for to allow for the two linking groups to remain associated with each other. Cognate linking groups may associate with each other covalently or non-covalently. An example of a suitable covalent linkage is the linkage shown in FIG. 4. An example of a suitable non-covalent linkage is an avidin-biotin linkage. In many instances, when cognate linking groups associate with each other non-covalently, their dissociation constants (Kd) will be at least 10⁻⁷.

As used herein, the term “close proximity”, when used in reference to donor nucleic acid and a target locus, refers to the local interaction environment of the target locus. This means that, when molecular motions (e.g., Brownian-like motion, intracellular fluid flows, etc.) are considered, the donor nucleic acid is close enough such that at least one portion of the donor nucleic acid is capable of touching nucleic acid at the target locus.

When a donor nucleic acid molecule is said to be brought into close proximity with a target locus by association with a nucleic acid cutting entity, the donor nucleic acid molecule will be (1) within a distance equal to the further portion of the nucleic acid cutting entity from the cut site, (2) within 300 angstroms, and/or (3) close enough such that the donor nucleic acid molecule is capable of contacting homologous nucleic acid at the target locus. Item (3) will vary with the length of the particular donor nucleic acid molecule. For example, one terminus of a donor nucleic acid may be linked to a portion of a nucleic acid cutting entity that is 200 angstroms for the target locus and the donor nucleic acid may be 600 angstroms in length. In such an instance, a substantial portion of the donor nucleic acid will be capable of contacting nucleic acid at and around the target locus. Double-stranded DNA molecules, for example, are about 3.4 angstroms in length for each base pair. Thus, a donor nucleic acid of 175 base pairs would be about 600 angstroms in length.

The invention thus includes compositions comprising nucleic acid cutting entities associated with donor nucleic acids, as well as methods for generating and using such compositions.

The number of donor nucleic acid molecules associated with each nucleic acid cutting entity may vary greatly and there are several ways to alter the number of donor nucleic acid molecules associated with each nucleic acid cutting entity. Some of those way are discussed here.

FIG. 1A shows a single donor nucleic acid molecule linked to one of two zinc finger-FokI fusion protein. FIG. 1B shows a pair of TAL-FokI fusion proteins designed to cut a target locus and donor nucleic acid molecules are linked to each member of the pair. Thus, in this instance, two donor nucleic acid molecules are brought into close proximity of the target locus by the nucleic acid cutting entity. FIG. 1D shows a CRISPR complex in which one donor nucleic acid molecule is linked to the guide RNA and two donor nucleic acid molecules are linked to the Cas9 protein. Collectively, these figures show methods by which one to three individual donor nucleic acid molecules may be brought into close proximity with target loci by association with nucleic acid cutting entities. In each instance, either each component of a nucleic acid cutting entity contains one donor nucleic acid molecule or each component of a nucleic acid cutting entity has a single donor nucleic acid molecule linked to each linking site. Thus, the invention includes methods by which more than one (e.g., from about 2 to about 20, from about 2 to about 10, from about 2 to about 5, from about 3 to about 10, from about 3 to about 6, from about 4 to about 12, from about 5 to about 10, etc.) donor nucleic acid molecule is brought into close proximity with a cut site generated by a nucleic acid cutting entity.

Multiple donor nucleic acid molecules may also be linked to single attachment sites. One technology that can be employed for this is dendrimer technology. Dendrimers may be used to attach multiple donor nucleic acid molecules to a single linking site of a nucleic acid cutting entity. In some such instances, donor nucleic acid molecules would typically be connected to a branched chemical entity and a single site on that chemical entity would also be linked to a one linking site of a nucleic acid cutting entity. Dendrimer products are sold by companies such as Glenn Research (Sterling, Va.) and Genisphere (Hatfield, Pa.).

The invention thus includes compositions in which from about 1 to about 200 (e.g., from about 1 to about 100, from about 1 to about 50, from about 1 to about 30, from about 1 to about 25, from about 1 to about 15, from about 1 to about 10, from about 1 to about 5, from about 1 to about 4, from about 1 to about 3, from about 1 to about 2, from about 2 to about 50, from about 2 to about 15, from about 2 to about 10, from about 2 to about 5, from about 2 to about 4, from about 4 to about 100, from about 4 to about 50, from about 4 to about 20, from about 4 to about 10, from about 4 to about 8, from about 6 to about 100, from about 6 to about 50, from about 6 to about 25, from about 6 to about 15, from about 6 to about 10, from about 8 to about 50, from about 8 to about 30, from about 8 to about 20, from about 10 to about 50, from about 10 to about 20, etc.) donor nucleic acid molecules are linked, on average, to each nucleic acid cutting entity. The invention further includes method for preparing and using such compositions (e.g., for homologous recombination reactions).

The number of donor nucleic acid molecules linked to a single linking site may also vary but with typically be from about 1 and to about 20 (e.g., from about 1 to about 15, from about 1 to about 10, from about 1 to about 5, from about 1 to about 3, from about 2 to about 15, from about 2 to about 6, from about 2 to about 4, from about 2 to about 3, from about 3 to about 8, from about 3 to about 20, etc.).

The invention relates, in part, to compositions and methods for increasing the number of donor nucleic acid molecules present near target loci. The invention further relates, in part, to compositions and methods for bringing one or more donor nucleic acid molecules in close proximity to target loci. These composition and methods relate, in part, to the use of nucleic acid cutting entities that have associated with them one or more donor nucleic acid molecule.

### Nucleic Acid Cutting Entities

As noted elsewhere herein, the invention relates, in part, to nucleic acid cutting entities associated with donor nucleic acid molecules. The association mechanism may be, for examples, covalent or non-covalent (e.g., hydrophobic, electrostatic, etc.).

In most instances, nucleic acid cutting entity components will be either proteins or nucleic acids but they may be cofactors and other associated molecules.

When a nucleic acid component of a nucleic acid cutting entity is associated with donor nucleic acid, the donor nucleic acid may be associated with any number of locations on the nucleic acid component. In many instances, one or more donor nucleic acid molecule will be associated with the 5′ or 3′ terminus. Using CRISPR systems for purposes of illustration, donor nucleic acid may be associated with the 5′ or 3′ terminus of crRNA, tracrRNA, and/or guide RNA. Typically, the association site will be chosen to eliminate or minimize loss of CRISPR nucleic acid functionality. Thus, if guide RNA is employed, then the association site on the guide RNA molecule will typically be chosen to minimize interference with cleavage activity of the nucleic acid cutting entity employing this guide RNA molecule.

One or more protein component of a nucleic acid cutting entity may also have associated with it one or more donor nucleic acid molecule. Association site selection will often be chosen to minimize expected and/or actual deleterious effects on nucleic acid cutting entity activity with respect to cutting activity at target loci. Using TAL effector for purposes of illustration, donor nucleic acid association sites that would be generally avoided would be in the repeat region that recognizes nucleic acid based upon sequence at target loci, functional nuclease active sites (e.g., RuvC and/or HNH domains, unless one of these site is inactivated as in “nicking” TAL effector proteins).

Proteins may contain linking that a naturally present linking site or an exogenously added one. An example of a naturally present linking site is a cysteine residue that is present in a naturally occurring protein that is a nucleic acid cutting entity or is a component of one. This includes a region of a protein (e.g., a segment of greater than about 20 amino acids) that is part of a protein that is a nucleic acid cutting entity or is a component of one. By way of example, many TAL-FokI fusions contain a large number of amino acids present in naturally occurring TAL effectors. Of course, non-naturally occurring TAL effectors can be designed and used to prepare nucleic acid cutting entities.

An exogenously added linking site is a linking site is a linking site that has been introduced in a nucleic cutting entity or a component of a nucleic acid cutting entity. This includes a linking site present in a non-naturally occurring protein produced by in silico design. One example, of an exogenously added linking site is avidin. Thus, the invention includes proteins of nucleic acid cutting entities that have linking sites associated with them, as well as nucleic acid cutting entities that are associated with donor nucleic acid molecules via such linking sites and methods for making and using such compounds.

Nucleic acid cutting entity proteins may have more than one (from about 2 to about 50, from about 2 to about 40, from about 2 to about 30, from about 2 to about 20, from about 2 to about 10, from about 4 to about 50, from about 4 to about 30, from about 4 to about 18, from about 8 to about 50, from about 8 to about 25, etc.) linking site associated with them. Further, these may be naturally present linking sites, exogenously added linking sites, or a mixture of these. In some instances, nucleic acid cutting entity proteins may have more than one (from about 2 to about 50, from about 2 to about 40, from about 2 to about 30, from about 2 to about 20, from about 2 to about 10, from about 4 to about 50, from about 4 to about 30, from about 4 to about 18, from about 8 to about 50, from about 8 to about 25, etc.) exogenously added linking site.

### Molecular Linking

A number of technologies may be used to link nucleic acid molecules to proteins and nucleic acid molecules to other nucleic acid molecules. Some of these means are by biotin-biotin binding protein interactions and Click-iT® reactions.

Proteins, for example, may associate with nucleic acid molecules by any number of means. Further, this association may be semi-random or site specific. By “semi-random” it is meant that the association may be at various locations of the protein. One example of this would be many methods for generating “metabolically” labeled protein containing linking sites that can be used to connect the protein to, for example, a donor nucleic acid molecule. A number of reagents useful for such labeled are available from, for example, Life Technologies and include Click-iT® AHA (L-azidohomoalanine) (Cat. No. C10102), Click-iT® HPG (L-homopropargylglycine) (Cat. No. C10186), Click-iT® farnesyl alcohol, azide (Cat. No. C10248), Click-iT® geranylgeranyl azide(Cat. No. C10249), Click-iT® fucose alkyne (tetraacetylfucose alkyne) (Cat. No. C10264), Click-iT® palmitic acid, azide (Cat. No. C10265), Click-iT® myristic acid, azide(Cat. No. C10268), Click-iT® GalNAz (tetraacetylated N-azidoacetylgalactosamine) (Cat. No. C33365), Click-iT® ManNAz (tetraacetylated N-azidoacetyl-D-mannosamine) (Cat. No. C33366), and Click-iT® GlcNAz (tetraacetylated N-azidoacetylglucosamine) (Cat. No. C33367).

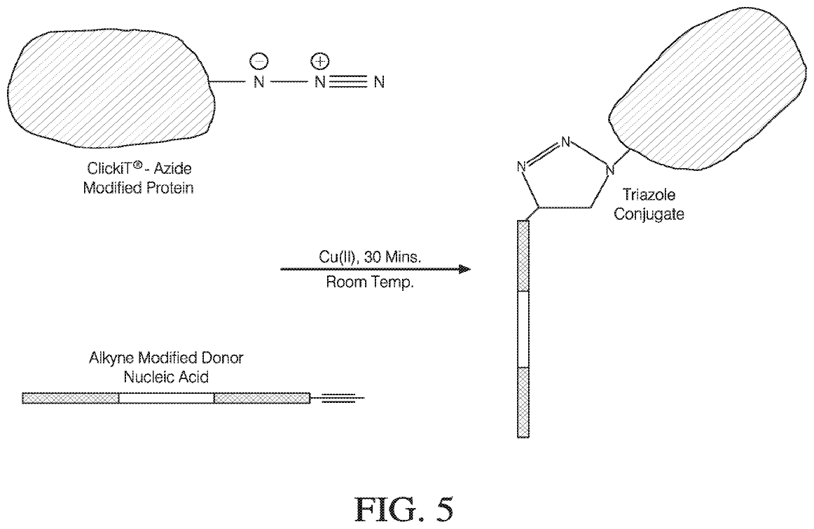

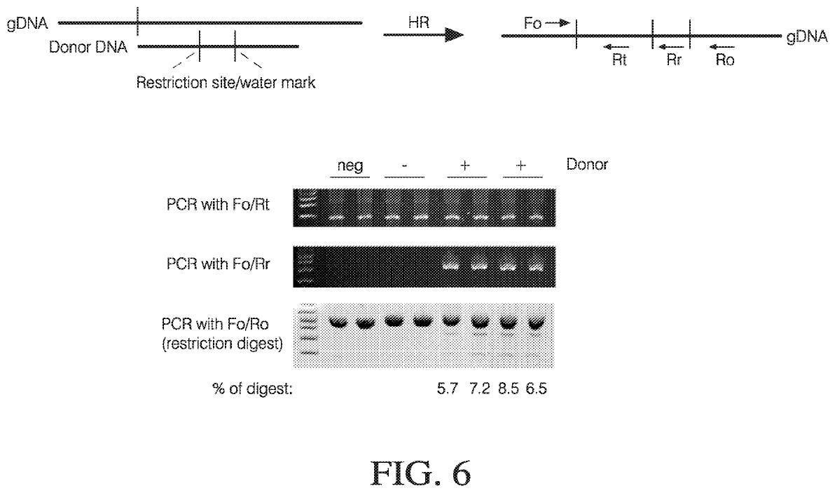

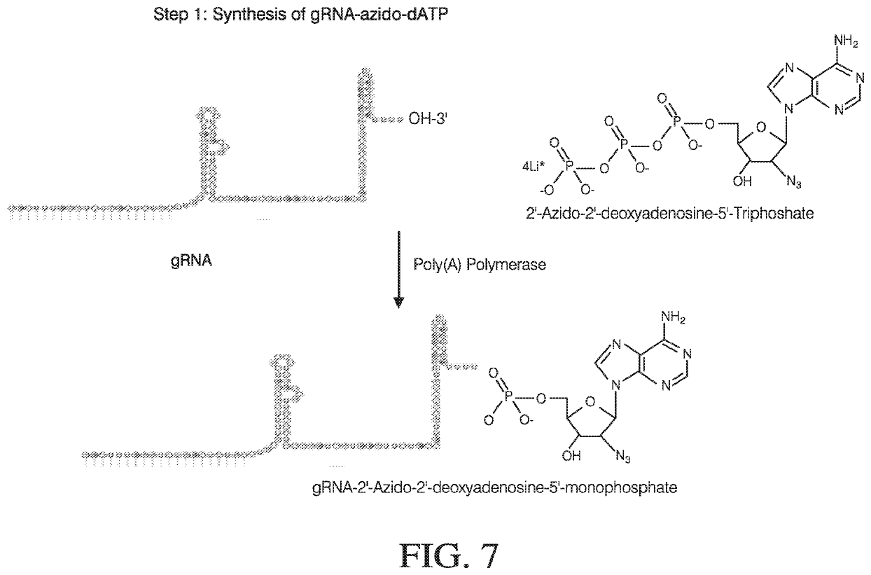

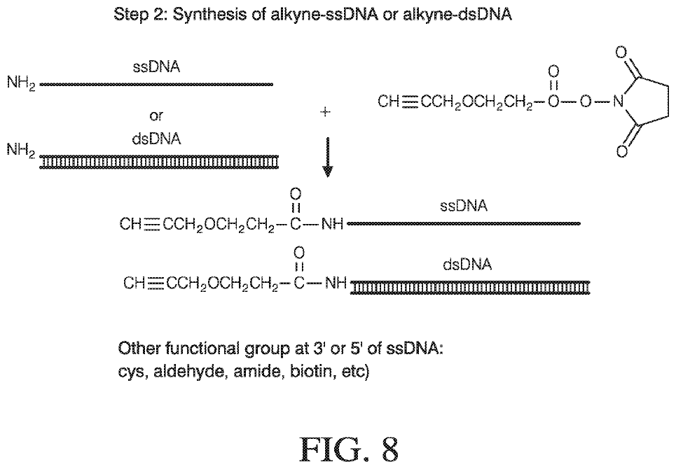

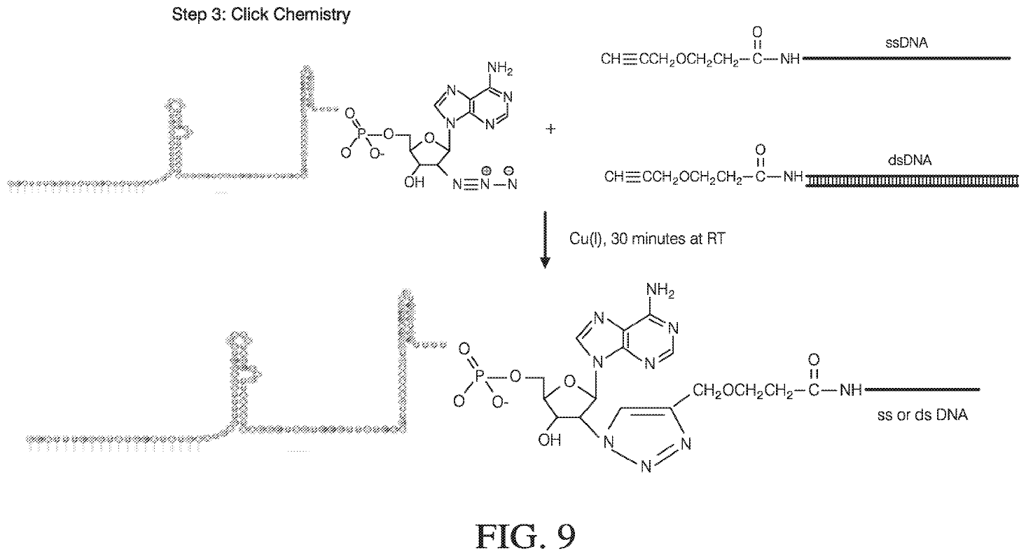

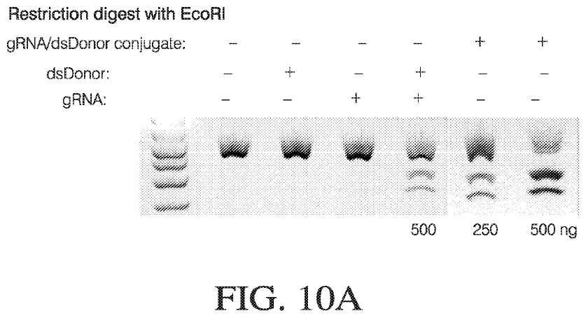

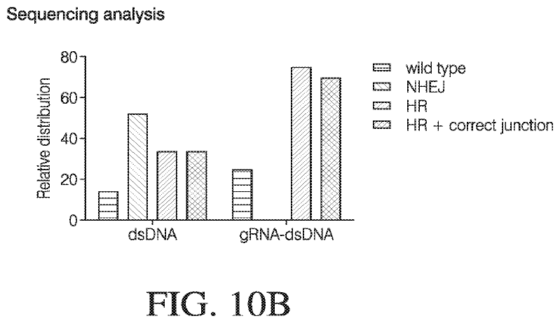

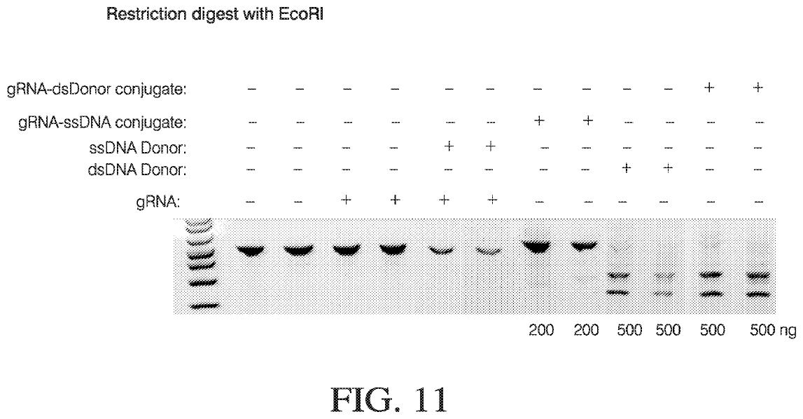

One example of linking of a protein to a nucleic acid molecule via Click-iT is shown in FIG. 5. In this instance, a reactive azide group is present on the protein and a reactive alkyne group is present on the nucleic acid molecules. Reaction in the presence of Cu(II) results in the formation of a triazole group connecting the two molecules.

“Metabolically” labeled proteins may be generated by production of the protein (e.g., intracellularly, via an in vitro transcription translation system, etc.) in the presence of compounds that are built into the polypeptide chain. They may also be produced by the use of protein group specific reagents (e.g., reagents that bind to sugar and lipid groups bound to proteins).

The interaction of biotin and avidin or streptavidin has been exploited for bind together proteins with nucleic acid detections. Because the biotin label is stable and small, it normally does not interfere with the function of labeled molecules.

Biotin is a vitamin that is present in small amounts in living cells. The valeric acid side chain of the biotin molecule can be derivatized in order to incorporate various reactive groups that facilitate the addition of a biotin tag to other molecules. Because biotin is relatively small (244.3 Daltons), it can be conjugated to many types of molecules, including nucleic acid molecules, often without significantly altering their biological activity.

Avidin is a protein derived from both avians and amphibians that shows considerable affinity for biotin. Avidin and other biotin-binding proteins, including streptavidin and deglycosylated avidin, have the ability to bind up to four biotin molecules.

Avidin is a biotin-binding protein that is believed to function as an antibiotic in the eggs of birds, reptiles and amphibians. Chicken avidin has a mass of 67,000-68,000 Daltons and is formed from four 128 amino acid-subunits, each binding one molecule of biotin. Avidin is highly glycosylated, with about 10% of its total mass being carbohydrate, contributing to its high solubility in water and aqueous salt solutions.

Avidin has a very high affinity for biotin molecules and is stable and functional over a wide range of pH and temperature. Avidin is amenable to extensive chemical modification with generally little to no effect on function, making it useful for the detection and protein purification of biotinylated molecules in a variety of conditions.

Streptavidin is a tetrameric biotin-binding protein that is isolated from Streptomyces avidinii and has a mass of 60,000 Daltons. While avidin and streptavidin have very little amino acid homology, their structures are very similar. Like avidin, streptavidin is thought to function as an antibiotic and has a very high affinity for biotin. Unlike avidin, streptavidin has no carbohydrate. Deglycosylated avidin (e.g., NeutrAvidin Protein, Thermo Fisher Scientific) is a 60,000 Dalton protein with low lectin binding activity.

The invention includes nucleic acid cutting entities (e.g., proteins) that contain one or more biotin binding region (e.g., composed of all or part of an avidin protein or protein with similar biotin binding activity).

Nucleic acid molecules (e.g., guide RNA and donor DNA) may be connected to each other in the practice of the invention may be produced by any number of means, including chemical synthesis. In some instances, nucleic acid molecules connected to each other may be produced by different methods. For example, a crRNA molecule produced by chemical synthesis may be connected to a tracrRNA molecule produced by in vitro transcription of DNA or RNA encoding the tracrRNA, followed by connection to a DNA donor nucleic acid molecule produced by PCR.

Another method that may be used to connect nucleic acid molecules is by “click chemistry” (see, e.g., U.S. Pat. Nos. 7,375,234 and 7,070,941, and US Patent Publication No. 2013/0046084, the entire disclosures of which are incorporated herein by reference). For example, one click chemistry reaction is between an alkyne group and an azide group (see FIG. 4). Any click reaction can be used to link nucleic acid molecules (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). Ligation of RNA molecules using a click chemistry reaction is advantageous because click chemistry reactions are fast, modular, efficient, often do not produce toxic waste products, can be done with water as a solvent, and can be set up to be stereospecific.

In one embodiment the present invention uses the “Azide-Alkyne Huisgen Cycloaddition” reaction, which is a 1,3-dipolar cycloaddition between an azide and a terminal or internal alkyne to give a 1,2,3-triazole for the ligation of nucleic acid molecules. One advantage of this ligation method is that this reaction can initiated by the addition of required Cu(I) ions.

Other mechanism by which nucleic acid molecules may be connected include the use of halogens (F—, Br—, I—)/alkynes addition reactions, carbonyls/sulfhydryls/maleimide, and carboxyl/amine linkages.

For example, an RNA molecule may be modified with thiol at 3′ (using disulfide amidite and universal support or disulfide modified support), and a DNA molecule may be modified with acrydite at 5′ (using acrylic phosphoramidite), then the two nucleic acid molecules can be connected by Michael addition reaction. This strategy can also be applied to connecting multiple nucleic acid molecules stepwise.

A number of additional linking chemistries may be used to connect nucleic acid molecules according to method of the invention. Some of these chemistries are set out in Table 1.

TABLE 1
Exemplary RNA Ligation Reactions

| Reaction | |
|---|---|
| Type | Reaction Summary |
| Thiol-yne | 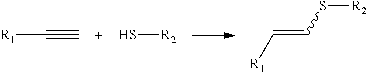 |
| NHS esters | 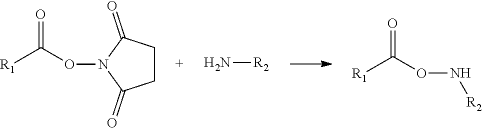 |
| Thiol-ene | 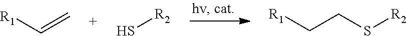 |
| Iso- cyanates | 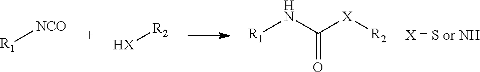 |
| Epoxy or aziridine | 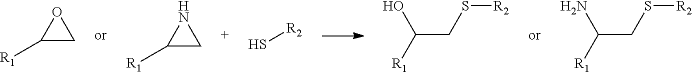 |
| Aldehyde- aminoxy | 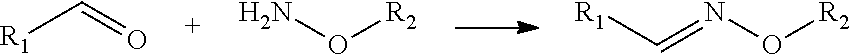 |
| Cu- catalyzed- azid- alkyne | 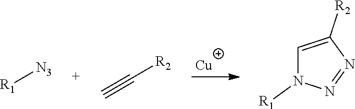 |
| Strain- promoted- azid- alkyne | 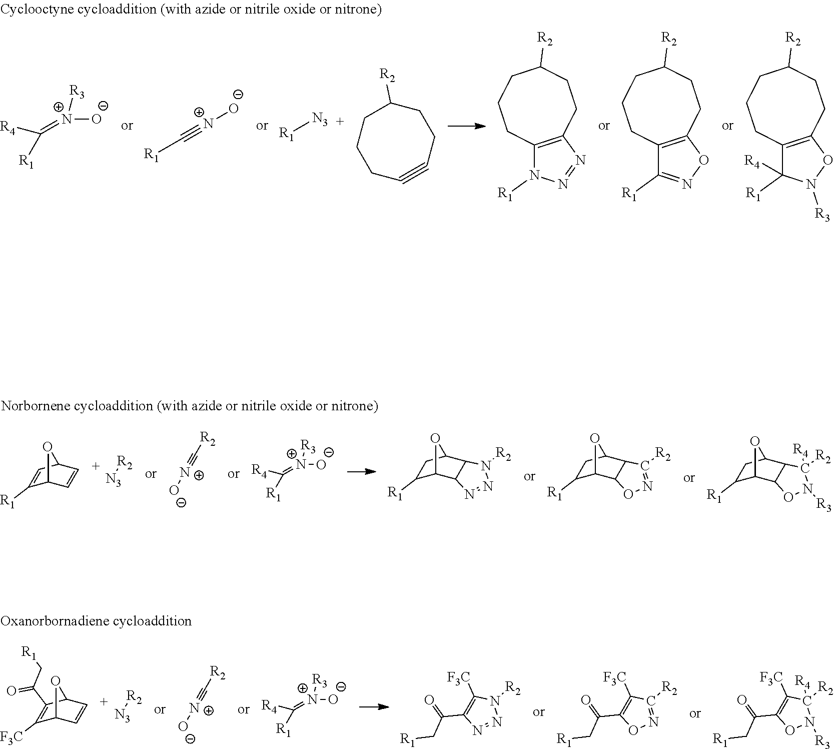 |
| Staudinger ligation | 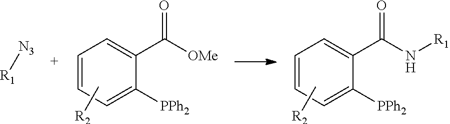 |
| Tetrazine ligation | 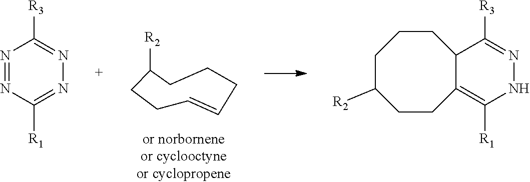 |
| Photo- induced tetrazole- alkene | 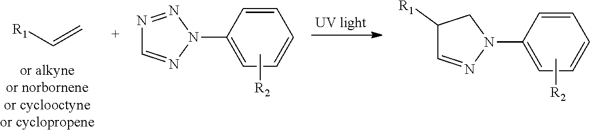 |
| [4 + 1] cyclo- addition | 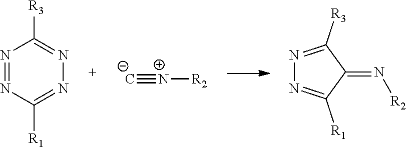 |
| Quadri- cyclane ligation | 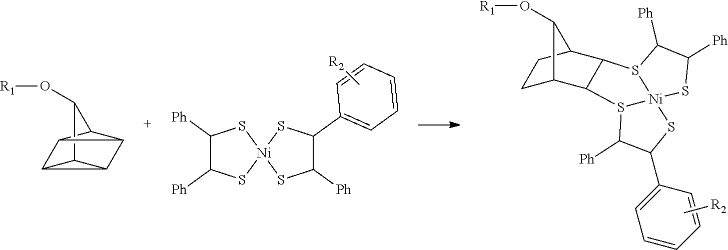 |

One issue with methods for linking nucleic acid molecules is that often they do not result in complete conversion of the segments to connected nucleic acid molecules. For example, some chemical linkage reactions only result in 50% of the reactants forming the desired end product. In such instances, it will often be desirable to remove reagents and unreacted nucleic acid molecules. This may be done by any number of means such as dialysis, chromatography (e.g., HPLC), precipitation, electrophoresis, etc. Thus, the invention includes compositions and method for linking nucleic acid molecules, where the reaction product nucleic acid molecules are separated from other reaction mixture components.

### Crispr Systems

CRISPR systems that may be used in the practice of the invention vary greatly. These systems will generally have the functional activities of a being able to form complex comprising a protein and a first nucleic acid where the complex recognizes a second nucleic acid. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In some embodiments, the CRISPR protein (e.g., Cas9) is derived from a type II CRISPR system. In specific embodiments, the CRISPR system is designed to acts as an oligonucleotide (e.g., DNA or RNA) -guided endonuclease derived from a Cas9 protein. The Cas9 protein for this and other functions set out herein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus desulforudis, Clostridium botulinum, Clostridium difficile, Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

### Introduction of Hr System Materials into Cells

The invention also includes compositions and methods for introduction of HR system components into cells. Introduction of a molecules into cells may be done in a number of ways including by methods described in many standard laboratory manuals, such as Davis et al., BASIC METHODS IN MOLECULAR BIOLOGY, (1986) and Sambrook et al., MOLECULAR CLONING: A LABORATORY MANUAL, 2nd Ed., Cold Spring Harbour Laboratory Press, Cold Spring Harbour. N.Y. (1989), such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

The invention includes methods in which different components of nucleic acid cutting entities are introduced into cells by different means, as well as compositions of matter for performing such methods. For example, a lentiviral vector may be used to introduce Cas9 coding nucleic acid operably linked to a suitable and guide RNA may be introduced by transfection. Further, donor nucleic acid may be associated with the guide RNA. Further Cas9 mRNA may be transcribed from a chromosomally integrated nucleic acid molecule, resulting in either constitutive or regulatable production of this protein.

In many instances, a single type of nucleic acid cutting entity molecule will be introduced into a cell but, particularly in instances where all nucleic acid cutting entities are not associated with donor nucleic acid, some nucleic acid cutting entity molecules may be expressed within the cell. One example of this is in the instance shown in FIG. 1A where two zinc finger-FokI fusions are used to generate a double-stranded break in intracellular nucleic acid. In this instance, only one of the zinc finger-FokI fusions is associated with a donor nucleic acid molecule. Thus, the other zinc finger-FokI fusion may be produced intracellularly.

Transfection agents suitable for use with the invention include transfection agents that facilitate the introduction of RNA, DNA and proteins into cells. Exemplary transfection reagents include TurboFect™ Transfection Reagent (Thermo Fisher Scientific), Pro-Ject™ Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.).

The invention further includes methods in which one molecule is introduced into a cell, followed by the introduction of another molecule into the cell. Thus, more than one nucleic acid cutting entity component may be introduced into a cell at the same time or at different times. As an example, the invention includes methods in which Cas9 is introduced into a cell while the cell is in contact with a transfection reagent designed to facilitate the introduction of proteins in to cells (e.g., TurboFect Transfection Reagent), followed by washing of the cells and then introduction of guide RNA while the cell is in contact with LIPOFECTAMINE™ 2000. One or both of these molecules may be associated with donor nucleic acid.

Conditions will normally be adjusted on, for example, a per cell type basis for a desired level of nucleic acid cutting entity component introduction into the cells. While enhanced conditions will vary, enhancement can be measure by detection of intracellular nucleic acid cutting activity. Thus, the invention includes compositions and methods for measurement of the intracellular introduction of nucleic acid cutting activity within cells.

With respect to CRISPRs, the invention also includes compositions and methods related to the formation and introduction of CRISPR complexes into cells.

A number of compositions and methods may be used to form CRISPR complexes. For example, cas9 mRNA and a guide RNA may be encapsulated in INVIVOFECTAMINE™ for, for example, later in vivo and in vitro delivery as follows. mRNA cas9 is mixed (e.g., at a concentration of at 0.6 mg/ml) with guide RNA. The resulting mRNA/gRNA solution may be used as is or after addition of a diluents and then mixed with an equal volume of INVIVOFECTAMINE™ and incubated at 50° C. for 30 min. The mixture is then dialyzed using a 50 kDa molecular weight curt off for 2 hours in 1X PBS, pH7.4. The resulting dialyzed sample containing the formulated mRNA/gRNA is diluted to the desire concentration and applied directly on cells in vitro or inject tail vein or intraperitoneal for in vivo delivery. The formulated mRNA/gRNA is stable and can be stored at 4° C.

For Cas9 mRNA transfection of cultured cells, such as 293 cells, 0.5 μg mRNA was added to 25 μl of Opti-MEM, followed by addition of 50-100 ng gRNA. Meanwhile, two μl of LIPOFECTAMINE™ 3000 or RNAiMax was diluted into 25 μl of Opti-MEM and then mixed with mRNA/gRNA sample. The mixture was incubated for 15 minutes prior to addition to the cells.

A CRISPR system activity may comprise expression of a reporter (e.g., green fluorescent protein, β-lactamase, luciferase, etc.) or nucleic acid cleavage activity. Using nucleic acid cleavage activity for purposes of illustration, total nucleic acid can be isolated from cells to be tested for CRISPR system activity and then analyzed for the amount of nucleic acid that has been cut at the target locus. If the cell is diploid and both alleles contain target loci, then the data will often reflect two cut sites per cell. CRISPR systems can be designed to cut multiple target sites (e.g., two, three four, five, etc.) in a haploid target cell genome. Such methods can be used to, in effect, “amplify” the data for enhancement of CRISPR system component introduction into cells (e.g., specific cell types). Conditions may be enhanced such that greater than 50% of the total target loci in cells exposed to CRISPR system components (e.g., one or more of the following: Cas9 protein, Cas9 mRNA, crRNA, tracrRNA, guide RNA, complexed Cas9/guide RNA, etc.) are cleaved. In many instances, conditions may be adjusted so that greater than 60% (e.g., greater than 70%, greater than 80%, greater than 85%, greater than 90%, greater than 95%, from about 50% to about 99%, from about 60% to about 99%, from about 65% to about 99%, from about 70% to about 99%, from about 75% to about 99%, from about 80% to about 99%, from about 85% to about 99%, from about 90% to about 99%, from about 95% to about 99%, etc.) of the total target loci are cleaved.

### KITS

The invention also provides kits for, in part, the preparation of nucleic acid cutting entities associated with donor nucleic acid molecules and use of such compounds for performing homologous recombination reactions (e.g., for editing of cellular genomes). As part of these kits, materials and instruction are provided for both the preparation of nucleic acid cutting entities and reaction mixtures.

Kits of the invention will often contain one or more of the following components:
1. One or more nucleic acid molecule encoding one or more component of a nucleic acid cutting entity (e.g., one or more TAL effector nuclease fusion, one or more zinc finger protein, one or more guide RNA, one or more CRISPR protein such as Cas9, dCas9, etc.),
2. One or more protein (e.g., one or more TAL effector nuclease fusion, one or more CRISPR protein such as Cas9, dCas9, etc.), and
3. One or more transfection reagent.

Kit reagents may be provided in any suitable container. A kit may provide, for example, one or more reaction or storage buffers. Reagents may be provided in a form that is usable in a particular reaction, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

### EXAMPLES

### Example 1: Highly Efficient Homologous Recombination in Human Genome Through CRISPR/Cas9 System

To maintain the integrity of human genome, homologous recombination (HR) is a very important pathway for repairing DNA damage in response to lesions in cells. For the past decades, significant amount of effort has been made to alter the non-homologous end joining (NHEJ) pathway to drive HR events, but the frequency of recombination in human genome remains extremely low of less than 1% and the reason is largely unknown. Recently, CRISPR/Cas9 systems have been developed that enable efficient genome editing by introduction of double-strand breaks at the target site of the genome, which is then repaired by either endogenous homologous recombination (HR) or (NHEJ). Unfortunately, the error-prone NHEJ pathway is predominant. Here it is shown that homologous recombination pathway in human cells is in fact highly efficient, depending on the local concentration of donor DNA. By increasing the concentration of a donor DNA or by conjugating a donor DNA to a guide RNA (gRNA), DNA repair can be driven almost exclusively towards homologous recombination pathway with efficiency of >75% in Jurkat T cells. This method is very useful in DNA repair of single nucleotide polymorphisms (SNPs) in cancer cells.

### Materials and Methods

Materials: Click-iT® Protein Reaction Buffer Kit, Alkyne Succinimidyl Ester, PureLink® PCR Micro Kit, PureLink® PCR Purification Kit, TranscriptAid™ T7 High Yield Transcription Kit, GeneArt® Genomic Cleavage Detection Kit, MEGAshortscript™ T7 Transcription Kit, MEGAclear™ Transcription Clean-Up Kit, Zero Blunt® TOPO® PCR Cloning Kit, PureLink® Pro Quick96 Plasmid Purification Kit, Qubit® RNA BR Assay Kit, Qubit® Protein Assay Kit, RPMI 1640 medium, Fetal Bovine Serum (FBS), Gibco® Human Episomal iPSC Line, Essential 8™ Medium, Geltrex®, GeneArt® Site-Directed Mutagenesis System, and AmpliTaq Gold® 360 Master Mix were from Thermo Fisher Scientific. Jurkat T cells were obtained from the American Type Culture Collection (ATCC®). 2′-Azido-2′-deoxyadenosine-5′-Triphosphate was purchased from TriLink®.

### Methods

### Preparation of Donor DNA

The genomic locus of HPRT was PCR-amplified by AmpliTaq Gold® 360 Master Mix using a forward primer 5′-acatcagcagctgttctg-3′ (SEQ ID NO: 1) and a reverse primer 5′-GGC TGA AAG GAG AGA ACT-3′ (SEQ ID NO: 2). The resulting 480 bp DNA fragment was then cloned into Zero Blunt® TOPO vector, followed by sequencing. Using GeneArt® Site-Directed Mutagenesis System, the crRNA target sequence catttctcagtcctaaaca GGG (SEQ ID NO: 3) within the DNA fragment was replaced by gaattccgttagtgtaggttctgacc ggg (SEQ ID NO: 4), in which a unique sequence and EcoRI restriction site were embedded. The regular donor DNA fragment containing the EcoRI restriction site was PCR-amplified using a pair of unmodified primers of 5′-acatcagcagctgttctg-3′ (SEQ ID NO: 1) and 5′-GGC TGA AAG GAG AGA ACT-3′ (SEQ ID NO: 2). On the other hand, the NH₂-modified donor DNA fragment was amplified using one unmodified forward or reverse primer in combination with one NH₂-modified reverse or forward primer respectively (5′-NH₂-acatcagcagctgttctg-3′ (SEQ ID NO: 1)/5′-GGC TGA AAG GAG AGA ACT-3′ (SEQ ID NO: 2) or 5′-acatcagcagctgttctg-3′ (SEQ ID NO: 1)/5′-NH₂-GGC TGA AAG GAG AGA ACT-3′ (SEQ ID NO: 2)). Also, the functional group, such as NH₂, can be located at either 5′ end or 3′ end of sense or antisense strand. Alternatively, a sense or antisense single strand DNA oligonucleotide: gaagaaggaactctagccagagtcttggaattccgttagtgtaggttctgaccgggtaatggactggggctgaatcacatg (SEQ ID NO: 5), which harbors a functional group at either 5′ end or 3′ end, such as NH₂, serves as donor for homologous recombination.

### In Vitro Transcription

The in vitro transcription of gRNA template was carried out using TranscriptAid T7 High Yield Transcription Kit. Briefly, 6 μl of the purified gRNA template (200-600 ng) was added to a reaction mixture containing 8 μl of NTP, 4 μl of 5× reaction buffer and 2 μl of T7 enzyme mix. The reaction was carried out at 37° C. for 2 hrs, followed by incubation with DNase I (1 units per 120 ng DNA template) for 15 minutes. The gRNA product was purified using MEGAclear™ Transcription Clean-Up kit as described in the manual. The concentration of RNA was determined using Qubit® RNA BR Assay Kit.

Synthesis of gRNA-azido-dATP

Three μg of gRNA was incubated for 1 hour at 37° C. with 2 mM azido-dATP in 50 μl of 1× Poly(A) Polymerase buffer containing 2.5 mM MnCl₂ and 20 units of Poly(A) Polymerase. The resulting gRNA-azido-dATP was then purified using MEGAclear™ Transcription Clean-Up Kit. The concentration of modified gRNA was estimated using NANODROP™.

### Synthesis of Alkyne-DNA

One mg of alkyne succinimidyl ester was dissolved in 100 μl of anhydrous DMSO to make up 10 mg/ml stock solution. One μl of stock solution was then added to 13 μg of 5′-amine-modified DNA fragment in 30 μl of 100 mM NaHCO₃. Alternatively, 1 nmoles of 80 bp ss DNA oligonucleotide was incubated with 4 μl of alkyne succinimidyl ester stock solution in 100 μl of 100 mM NaHCO₃. The reaction was carried out for 4 hours at room temperature. The alkyne-modified DNA fragment or alkyne-modified ss DNA oligonucleotide was then purified using PureLink® PCR Purification Kit. The concentration was measured using NANODROP™.

Synthesis of gRNA and DNA Conjugate-Click Reaction

50 pmoles of gRNA-azido-dATP was mixed with 50 pmoles of alkyne DNA fragment or alkyne ss DNA oligonucleotide, followed by addition of H₂O to a total volume of 60 μl. 100 μl of 2×reaction buffer was added, followed by addition of 10 μl of CuSO₄ solution. The sample was vortexed for 5 seconds. 10 μl of Additive 1 was then added to the sample and incubated for 2-3 minutes at room temperature. Finally 20 μl of Additive 2 was added. After vortexing for 5 seconds, the sample was incubated for 20 minutes at room temperature. The gRNA-DNA conjugate was then purified using PureLink® PCR Micro Kit. The concentration was determined by NANODROP™.

### Transfection via Electroporation

Jurkat T cells were maintained in RPMI medium. Gibco Episomal iPSCs were cultured in E8 essential medium on Geltrex-coated plates. For Jurkat T cells, 2×10⁵ cells were used per electroporation using Neon® Transfection System 10 μL Kit (Thermo Fisher Scientific) with pulse voltage set at 1700 volts, pulse width at 20 ms and number of pulse at one. On the other hand, 1×10⁵ iPSCs were used per electroporation with 1100 Volts, 20 ms and 1 pulse. 1.5 to 2.0 μg of purified Cas9 protein was preincubated for 10 minutes at room temperature with 300 to 400 ng of gRNA in 10 μl of Resuspension Buffer R provided in the kit. Prior to electroporation, 1 μl of 1 nmole/μl unmodified ss DNA oligonucleotide or 500 ng/μl of ds donor DNA fragment was added. Samples without donor DNA or gRNA were used as controls. Alternatively, 1.5 to 2.0 μg of purified Cas9 protein was incubated for 10 minutes with 2 μl of 100 ng of gRNA-ssDNA oligo conjugate or 250 ng/μl of gRNA-dsDNA conjugate. Meanwhile, the cells were counted and aliquots of cells were transferred to a sterile test tube, followed by centrifugation at 2000 rpm for 5 minutes. The supernatant was aspirated and the cell pellet was resuspended in 1 ml of PBS without Ca²⁺and Mg²⁺. Upon centrifugation, the supernatant was carefully aspirated so that almost all the PBS buffer was removed with no or minimum loss of cells. Samples, prepared as described above, were used to resuspend the cell pellets. The electroporated cells were transferred immediately to a 24 well containing 0.5 ml of the corresponding growth medium without dipping the tip into the medium, followed by incubation for 48 hrs in a humidified 5% CO₂ incubator.

### Quantitation of Homologous Recombination

Upon incubation for 48 hours, the cells were harvested by centrifugation and then washed once with PBS. The cell lysate was PCR amplified with AmpliTaq Gold® 360 Master Mix using a forward primer of 5′-acatcagcagctgttctg-3′ (SEQ ID NO: 1) and a reverse primer of 5′-CAT GCA TAG CCA GTG CTT GAG AAG-3′ (SEQ ID NO: 6). The reverse primer is located at the genome outside of the recombination region. The PCR product was digested with EcoRI restriction enzyme or directly cloned into Zero Blunt® TOPO® vector. 96 of colonies were randomly picked for sequencing.

### RESULTS AND DISCUSSION

Previously we demonstrated that the delivery of Cas9 protein/gRNA complexes is sufficient to introduce double-strand breaks in human genome with more than 90% cleavage efficiency. However, it was found that the damaged DNAs are repaired primarily by non-homologous end joining pathway. To examine the efficiency of homologous recombination, we constructed a double-strand donor DNA fragment harboring an EcoRI restriction site and a unique sequence for PCR amplification. Alternatively, an 80 bp single-strand DNA oligonucleotide was used. The double-stranded DNA (dsDNA) donor or single-stranded DNA (ssDNA) donor was then co-transfected with Cas9 protein/gRNA complexes into Jurkat T cells via electroporation. Upon 48 hours post transfection, the cells were lysed and the target sequences at the genomic loci were PCR-amplified, followed by analysis of restriction digestion and sequencing. Initial test with 100-200 ng donor DNA resulted in very low homologous recombination efficiency. To boost recombination events, we increased the amount of donor DNA to 500 ng per reaction or coupled the donor DNA to a gRNA through Click chemistry. To our surprise, the recombination efficiency significantly increased with the increase of donor DNA with 34% in Jurkat T cells according to sequencing analysis. When a donor DNA was conjugated to a gRNA, the recombination efficiency increased to 75% in Jurkat T cells. Furthermore, the NHEJ pathway was completely inhibited when a donor DNA was coupled to a RNA in Jurkat T cells, whereas the NHEJ pathway was still competing with HR pathway when non-conjugated DNA fragment was delivered. These results indicated that the mammalian cells have all the cellular machinery to carry out homologous recombination depending on availability of the donor in close proximity.

While the foregoing embodiments have been described in some detail for purposes of clarity and understanding, it will be clear to one skilled in the art from a reading of this disclosure that various changes in form and detail can be made without departing from the true scope of the embodiments disclosed herein. For example, all the techniques, apparatuses, systems and methods described above can be used in various combinations.

1-20. (canceled)

21. A composition comprising a CRISPR guide RNA molecule and a donor nucleic acid molecule, wherein the donor nucleic acid molecule is covalently bound to the CRISPR guide RNA molecule.

22. The composition of claim 21, wherein the donor nucleic acid molecule is covalently bound to the 3′ terminus of the CRISPR guide RNA molecule.

23. The composition of claim 21, further comprising a transfection reagent.

24. The composition of claim 21, wherein the donor nucleic acid molecule has at least one region of sequence homology to a target locus present in a cell.

25. The composition of claim 24, wherein the region of sequence homology to the target locus is located at one of both termini to the donor nucleic acid.

26. A composition comprising (1) a nucleic acid molecule encoding a Cas9 protein and (2) a donor nucleic acid molecule, wherein the donor nucleic acid molecule is covalently bound to a terminus of a CRISPR guide RNA molecule.

27. The composition of claim 26, wherein the donor nucleic acid molecule is covalently bound to the 3′ terminus of the CRISPR guide RNA molecule.

28. The composition of claim 26, wherein the Cas9 protein has nickase activity.

29. The composition of claim 26, wherein the RNA nucleic acid encoding a Cas9 protein has mutations in the HNH and RuvC domains.

30. The compositions of claim 26, wherein the Cas9 protein further comprises an NLS.


SEQUENCE LISTING (7 sequences)

SEQ ID NO: 1 - DNA, 18 bp, synthetic construct
  misc_feature 1..18: note = source = /note="Description of Artificial Sequence: Synthetic primer"
  source 1..18: mol_type = other DNA
acatcagcagctgttctg

SEQ ID NO: 2 - DNA, 18 bp, synthetic construct
  misc_feature 1..18: note = source = /note="Description of Artificial Sequence: Synthetic primer"
  source 1..18: mol_type = other DNA
ggctgaaaggagagaact

SEQ ID NO: 3 - DNA, 22 bp, synthetic construct
  misc_feature 1..22: note = source = /note="Description of Artificial Sequence: Synthetic oligonucleotide"
  source 1..22: mol_type = other DNA
catttctcagtcctaaacaggg

SEQ ID NO: 4 - DNA, 29 bp, synthetic construct
  misc_feature 1..29: note = source = /note="Description of Artificial Sequence: Synthetic oligonucleotide"
  source 1..29: mol_type = other DNA
gaattccgttagtgtaggttctgaccggg

SEQ ID NO: 5 - DNA, 81 bp, synthetic construct
  misc_feature 1..81: note = source = /note="Description of Artificial Sequence: Synthetic oligonucleotide"
  source 1..81: mol_type = other DNA
gaagaaggaactctagccagagtcttggaattccgttagtgtaggttctgaccgggtaatggactggggctgaatcacatg

SEQ ID NO: 6 - DNA, 24 bp, synthetic construct
  misc_feature 1..24: note = source = /note="Description of Artificial Sequence: Synthetic primer"
  source 1..24: mol_type = other DNA
catgcatagccagtgcttgagaag

SEQ ID NO: 7 - RNA, 104 nt, synthetic construct
  misc_feature 1..104: note = source = /note="Description of Artificial Sequence: Synthetic polynucleotide"
  source 1..104: mol_type = other RNA
gcatttcggtatgcatatgaatgagttttagagctagaaatagcaagttaaaataaggctagtccgttatcaacttgaaaaagtggcaccgagtcggtgctttt